(19) United States
(12) Patent Application Publication
(10) Pub. No.: US 2021/0259911 A1
(43) Pub. Date: Aug. 26, 2021

(54) Massaging Apparatus for Wrapping Around Abdomen

(71) Applicant: Joshua Makaio Earsley, Anaheim, CA (US)

(72) Inventor: Joshua Makaio Earsley, Anaheim, CA (US)

(21) Appl. No.: 16/801,518

(22) Filed: Feb. 26, 2020

(51) Int. Cl. A61H 11/00; A61H 1/00; A61F 7/02; A61H 9/00

(52) U.S. Cl. CPC A61H 11/00; A61H 1/00; A61F 7/02; A61H 9/0078; A61H 2011/005; A61H 2201/5025; A61H 2201/163; A61H 2201/1652; A61H 2201/5097; A61H 2201/1207; A61H 2201/0207

(57) ABSTRACT

An abdomen massaging system massages through selective application of heat, vibrations, and pressure the muscles of the abdomen for relieving menstrual pains and cramps. The system utilizes a massage belt that is wearable about the pelvis. The massage belt has a fastener, such as straps, that hold the body portion around abdomen. The massage belt is remotely regulated by a control unit or a remote controller to selectively generate vibration with a vibratory element, heat with a heat element, and pressure with a pressure element. These massaging components are controlled by a hands-free control unit that initiates a vibration signal, a heat signal, and a pressure signal to the massage belt. The heat element, vibratory element, and pressure element provide a perceptible stimulus to the pelvic area at predetermined times and intensities. The control unit regulates signals between a rest mode, a signal mode, a timer, and a speed control.

### CROSS REFERENCE OF RELATED APPLICATIONS

This application claims the benefits of U.S. provisional application No. 62/859,611, filed Jun. 10, 2019 and entitled MASSAGING APPARATUS FOR WRAPPING AROUND ABDOMEN, which provisional application is incorporated by reference herein in its entirety.

### FIELD OF THE INVENTION

The present invention relates generally to an abdomen massaging system and method of operation. More so, the present invention relates to a massaging system that massages the abdomen, and specifically the lower abdomen and lower back, through selective application of heat, vibrations, and pressure the muscles of the abdomen to relieve menstrual pains and cramps through use of a massage belt wearable about the pelvis, and controlled by a hands-free control unit, and operable to initiate in the massage belt: a vibration signal, a heat signal, and a pressure signal, with the signals regulated between a rest mode, a signal mode, a timer, and a speed control.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, women suffer from menstrual cramps and pains in the pelvic area. A common disorder called Dysmenorrhea causes severe pains during their cycle. These symptoms occur as a result of congestion in the pelvic area. Women have used a variety of girdles and garments for releasing the menstrual pains. However, none have been particularly effective for relieving pain and cramping in the pelvic area. For example, in the art of applying fixed pressure to the body, a number of devices, primary belts, have been proposed which operate on the back by applying pressure there.

Other proposals have involved devices for massaging the body. The problem with these gripping devices is that they do not specifically massage the pelvic region, so as to assist a woman during menstruation. Also, heat, pressure, and vibrations are not selectively used to create a desired massaging effect on the pelvic region. Even though the above cited massaging devices meets some of the needs of the market, a hands-free abdomen massaging system and method of operation that massages through selective application of heat, vibrations, and pressure the muscles of the abdomen to relieve menstrual pains and cramps; and utilizes a massage belt wearable about the pelvis, and controlled by a hands-free control unit, and operable to initiate in the massage belt: a vibration signal, a heat signal, and a pressure signal, is still desired.

### SUMMARY

Illustrative embodiments of the disclosure are generally directed to a hands-free abdomen massaging system and method of operation. The massaging system serves to train and strengthen the muscles of the abdomen, and specifically the lower abdomen and lower back, for relieving menstrual pains and cramps. The system allows the elements of heat, pressure, and vibrations to be selectively used to create a desired massaging effect on the pelvic region. The system utilizes a massage belt that is wearable about the pelvis. The massage belt has at least one fastener, such as opposing straps, that hold the body portion around the abdomen.

The massage belt is remotely regulated by a control unit or a remote controller to selectively generate vibration with a vibratory element, heat with a heat element, and pressure with a pressure element. These massaging components are controlled by a hands-free control unit that initiates a vibration signal, a heat signal, and a pressure signal to the massage belt. The heat element, vibratory element, and pressure element provide a perceptible stimulus to the pelvic area at predetermined times and intensities. The control unit regulates the signals between a rest mode, a signal mode, a timer, and a speed control.

In one aspect, an abdomen massaging system, comprises:
a massage belt operable to be worn on, or proximal to, the pelvic area, the massage belt comprising a heat element operable to generate heat, the massage belt further comprising a vibratory element operable to generate vibrations, and a pressure element operable to generate pressure;
a control unit operable to regulate the heat element, the vibratory element, and the pressure element,
the control unit comprising a signal transmission device and a signal reception device, the signal transition device transmitting a heat signal to the heat element to initiate heat in the massage belt, the heat signal comprising a heat signal mode and a heat rest mode, the heat signal mode initiating operation of the heat element, the heat rest mode terminating operation of the heat element,
the control unit transmitting a vibration signal to the vibratory element to initiate vibration in the massage belt, the vibration signal comprising a vibration signal mode and a vibration rest mode, the vibration signal mode initiating operation of the vibratory element, the vibration rest mode terminating operation of the vibratory element
the control unit transmitting a pressure signal to the pressure element to initiate pressure in the massage belt, the pressure signal comprising a pressure signal mode and a pressure rest mode, the pressure signal mode initiating operation of the pressure element, the pressure rest mode terminating operation of the pressure element; and
a remote controller in operational communication with the signal reception device of the control unit, the remote controller operable to command the control unit to generate the heat signal, the vibration signal, and the pressure signal.

In another aspect, the control unit is on a control side of the massage belt, while the elements are disposed on a massage side of the massage belt.

In another aspect, the heat element comprises a heating pad.

In another aspect, the vibratory element comprises a vibrator.

In another aspect, the pressure element comprises a contraction strap or an air pressure bladder.

In another aspect, the pressure element applies pressure at a base line pressure.

In another aspect, the pressure element is operative when the pressure applied by the pressure module is below the base line pressure.

In another aspect, the pressure signal mode is operative when the pressure applied by the pressure module is above the base line pressure.

In another aspect, the pressure rest mode is operative below the base line pressure.

In another aspect, the control unit comprises a speed level controller, the speed level controller being operational to regulate the intensity of the vibratory element and the pressure element.

In another aspect, the speed level controller comprises a high level, a medium level, and a slow level.

In another aspect, the control unit comprises a timer, the timer regulating the duration of the signals.

In another aspect, the timer regulating the signals for a predetermined duration.

In another aspect, the control unit comprises a recharge outlet operable to receive power from an external power source, the recharge outlet being in electrical communication with the heat element, the vibratory element, and the pressure element.

In another aspect, further comprising a power cord carrying power from the external power source to the recharge outlet.

In another aspect, the control unit comprises an idle module and a constant module, the idle module being operable to terminate operation of the vibratory element, the heat element, and the pressure element.

In another aspect, the massage belt comprises a body and at least one fastener, the fastener being operable to help retain the body on, or proximal to, the pelvic area.

In another aspect, the at least one fastener comprises a pair of straps arranged on the one opposing ends of the body portion to fasten the body portion on the pelvic area.

In another aspect, the at least one fastener comprises a snap button.

In another aspect, the remote controller includes at least one of the following: a cell phone, a radio remote controller, and an iPad.

One objective of the present invention is to stimulate the pelvic area with heat, vibrations, and pressure applied at different intervals and intensities.

Another objective is to produce a hands-free massaging device for relieving menstrual pains and cramps.

Another objective is to remotely control the massage belt.

Yet another objective is to secure the massage belt to the pelvic area with straps.

Yet another objective is to provide a speed controller to regulate the vibrations and pressure.

Yet another objective is to massage and relieve the abdomen, lower abdomen, and lower back.

Yet another objective is to provide an inexpensive to manufacture abdomen massaging system.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a top view of an exemplary abdomen massaging system, showing the massage belt, in accordance with an embodiment of the present invention;

FIG. 2 illustrates a top view of an exemplary abdomen massaging system, showing the control unit and the remote controller, in accordance with an embodiment of the present invention;

FIG. 3 illustrates a perspective view of the abdomen massaging system wrapped around the lower abdomen, in accordance with an embodiment of the present invention;

FIG. 4 illustrates a perspective view of the abdomen massaging system, showing the massage belt, in accordance with an embodiment of the present invention;

FIG. 5 illustrates a perspective view of the abdomen massaging system, showing the control unit, in accordance with an embodiment of the present invention;

FIG. 6 illustrates a top view an alternative view of an abdomen massaging system, in accordance with an embodiment of the present invention;

FIGS. 7A and 7B illustrate a top view and a side view of an exemplary interior wrap, in accordance with an embodiment of the present invention;

FIGS. 8A and 8B illustrate a top view and a side view of an exemplary exterior wrap, in accordance with an embodiment of the present invention;

FIGS. 9A and 9B illustrate a top view and a side view of an exemplary strap, in accordance with an embodiment of the present invention;

FIGS. 10A and 10B illustrate a top view and a side view of an exemplary pocket, in accordance with an embodiment of the present invention;

FIGS. 11A and 11B illustrate a top view and a side view of an exemplary hook and loop fastener that attaches to ends of strap, in accordance with an embodiment of the present invention; and

FIGS. 12A and 12B illustrate a top view and a side view of an exemplary case, in accordance with an embodiment of the present invention.

Like reference numerals refer to like parts throughout the various views of the drawings.

### DETAILED DESCRIPTION OF THE INVENTION

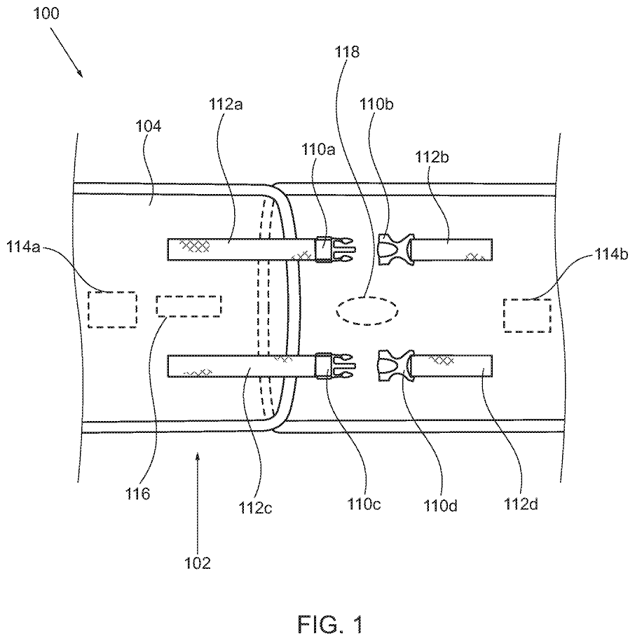

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

An abdomen massaging system 100 is referenced in FIGS. 1-12B. A hands-free abdomen massaging system 100, hereafter “system 100” provides a massage belt 102 that forms a snug fit to the pelvic area, lower abdomen, or proximal thereto. The massage belt 102 applies multiple massaging forces, including heat, vibrations, and pressure directly on the pelvic area 300, or in proximity to the pelvic area 300, and specifically the lower abdomen and lower back, depending on the position of the massage belt 102. The heat, pressure, and vibrations are selectively used to create a desired massaging effect on the pelvic area 300. The system 100 utilizes a unique control unit 200 that transmits signals to the massage belt 102 for operation, and terminating operations, for heating, vibrating, and pressurizing the massage belt 102. The control unit 200 applies various modes, speed levels, and time durations to the massage belt 102. Further, a remote controller 204 can regulate the massaging elements available on the massage belt 102.

As referenced in FIG. 1, the system 100 provides a massage belt 102, which is worn directly on, or in proximity to the pelvic area 300 for applying heat, vibrations, and pressure thereto. Those skilled in the art will recognize that menstrual cramps and pains occur at the pelvic area. Thus, the massage belt 102 is applied directly onto this region, so as to massage and reduce pains thereto. In some embodiments, the massage belt 102 may include a body portion 104 and at least one fastener 110a-d. The body portion 104 is a section of fabric sized to substantially cover the pelvic area.

The heat, vibratory, and pressure elements 114a, 114b, 116, 118 may be attached to, or integrated into the body portion 104 for creating the desired massaging effect therethrough. The fastener 110a-d is operable to help retain the body portion 104 on, or proximal to, the pelvic area. As FIG. 1 illustrates, the at least one fastener 110a-d comprises a pair of straps 112a-d arranged on the one opposing ends of the body portion 104 to fasten the body portion 104 on the pelvic area. In some embodiments, the at least one fastener 110a-d may include, without limitation, a snap button, a hook and loop fastener, a magnet, and an adhesive.

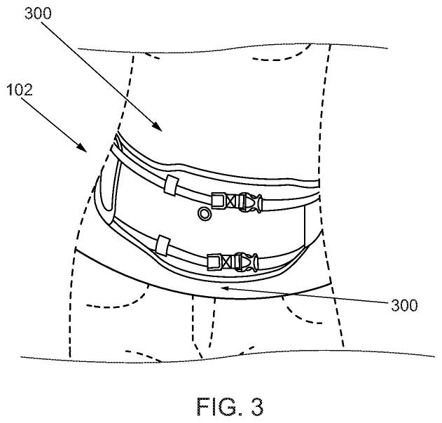

The massage belt 102 is configured to be worn on, or proximal to, the pelvic area 300. For example, FIG. 3 illustrates a perspective view of the abdomen massaging system wrapped around the lower abdomen. This is the affected region of menstrual pain and cramps. The massage belt 102 comprises a heat element 114a, 114b operable to generate heat. Those skilled in the art will recognize that localized application of heat causes vessels to dilate, which can help reduce pain. In one non-limiting embodiment, the heat element 114a, 114b comprises a heating pad. The heat element 114a, 114b may include an insulated wire or heating element inserted into a fabric that heats when it is plugged. However, other heating elements known in the art that generate heat when electricity is applied may also be used.

In other embodiments, the massage belt 102 further comprising a vibratory element 116 configured to generate vibrations, and thereby massage the effected area on or near the pelvic region. In one non-limiting embodiment, the vibratory element 116 is a device with switch contacts mounted at the ends of flexible metal strips. The strips can be vibrated by an electromagnet causing the contacts to open and close rapidly. However, in other embodiments, different types of vibratory mechanisms known in the art may also be used.

In other embodiments, the massage belt 102 also provides a pressure element 118 that is operable to generate pressure. The pressure is applied directly on the abdomen, which serves as a massaging effect. This creates a deep tissue massage effect, as known in the art. In one non-limiting embodiment, the pressure element 118 is a contraction strap or an air pressure bladder. However, in other embodiments, different types of pressure mechanisms known in the art may also be used.

To regulate the massage belt 102, the system 100 provides a control unit 200 that is configured with multiple electrical connections, switches, and signal reception/transmission components for communicating commands to the massage belt 102. In one possible embodiment, the control unit 200 is operable to regulate the heat element 114a, 114b, the vibratory element 116, and the pressure element 118.

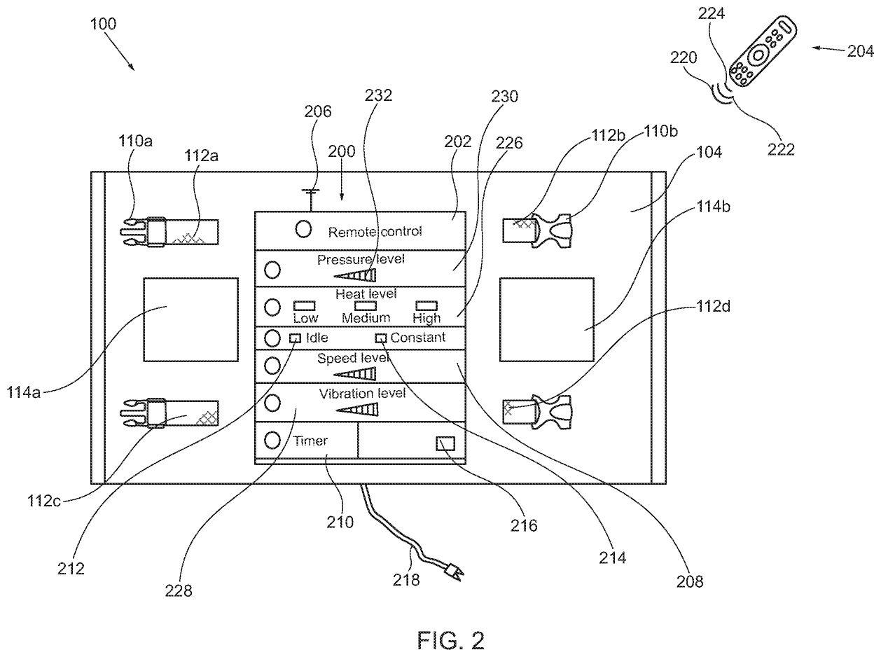

FIG. 2 illustrates an exemplary control unit 200 which is integrated directly into the body portion 104 of the massage belt 102. However, in other embodiments, the control unit 200 may be separate from the massage belt 102. In yet another possible embodiment, the control unit 200 may be on a control side 108 of the belt, while the elements 114a, 114b, 116, 118 are disposed on a massage side 106 of the belt.

In some embodiments, the control unit 200 comprises a signal transmission device 206 for transmitting the signals to the massage belt 102. However, in embodiments where the control unit 200 is integral with the body portion 104 of the massage belt 102, the signal transmission device 206 may not be necessary. The control unit 200 may also include a signal reception device 202 for receiving signals/commands from the remote controller 204. In some embodiment, the signal may be defined, without limitation, as a radio signal, an infra-red signal, and a Wi-Fi signal. Various transistors, receivers, and other signal processing equipment known in the art may be used.

In one embodiment, the signal transition device 206 of the control unit 200 is configured to transmit a heat signal 220 to the heat element 114a, 114b to initiate heat in the massage belt 102. The heat signal 220 comprises a heat signal mode and a heat rest mode regulated through a heat module 226 on the control unit 200. The heat signal mode initiates operation of the heat element 114a, 114b. The heat rest mode terminates operation of the heat element 114a, 114b. The signal and rest modes are easily accessed and selected by the heat module 226.

In another embodiment, the signal transition device 206 of the control unit 200 is configured to transmit a vibration signal 222 to the vibratory element 116 to initiate vibration in the massage belt 102. The vibration signal 222 comprises a vibration signal mode and a vibration rest mode regulated through a vibration module 228 on the control unit 200. The vibration signal mode initiates operation of the vibratory element 116. The vibration rest mode terminates operation of the vibratory element 116. The signal and rest modes are easily accessed and selected by the vibration module 228.

In yet another embodiment, the signal transition device 206 of the control unit 200 is configured to transmit a pressure signal 224 to the pressure element 118 to initiate pressure in the massage belt 102. The pressure signal 224 comprises a pressure signal mode and a pressure rest mode regulated through a pressure module 230 on the control unit 200. The pressure signal mode initiates operation of the pressure element 118. The pressure rest mode terminates operation of the pressure element 118. The signal and rest modes are easily accessed and selected by the pressure module 230.

In one embodiment, the pressure element 118 applies pressure at a base line pressure 232. Thus, the pressure element 118 is actuated into operative when the pressure applied by the pressure module is below the base line pressure 232. In one possible embodiment, the pressure signal mode is operative when the pressure applied by the pressure module is above the base line pressure 232. However, in other embodiments, the pressure rest mode is made operational below the base line pressure 232. The base line pressure 232 may be defined by a pressure that is applied, which is sufficient for deep tissue massage, without causing excessive pain or tissue damage, i.e., bruising.

Looking again at FIG. 2, the control unit 200 comprises a speed level controller 208 that is configured to regulate the intensity of the vibratory element 116 and the pressure element 118. In some embodiments, the speed lever controller 208 may be operable through precise regulation of electrical output, voltage, current, and the like. The speed level controller 208 is designed to alternately transitional between the strength of the vibration element and the pressure-exerting element. In one non-limiting embodiment, the speed level controller 208 comprises a high level, a medium level, and a slow level. The high-level speed is more intense and requires greater power than the low speed, for example.

In another embodiment, the control unit 200 comprises a timer 210. The timer 210 regulates the duration of the signals and the amount of time each heat/vibration/pressure mode is utilized for a massage session, for example. In another embodiment, the timer 210 regulates the signals for a predetermined duration. For example, the vibration signal 222 may be activated for 30 seconds, and then alternatively, the pressure signal 224 is activated for 30 seconds. This time-based element-selection system creates a customized massaging effect on the pelvic region.

In yet another possible embodiment of the regulation by the control unit 200, the control unit 200 comprises an idle module 212 and a constant module 214. The idle module 212 is operable to terminate operation of the vibratory element 116, the heat element 114a, 114b, and the pressure element 118. The constant module 214 is configured to initiate operation of the vibratory element 116, the heat element 114a, 114b, and the pressure element 118 at predetermined intensities and speeds. The idle module 212 and the constant module 214 can be selectively switched through the control unit 200 or remote controller 204.

In yet another embodiment, the control unit 200 comprises a recharge outlet 216 that helps power at least one of: the heat element 114a, 114b, the vibratory element 116, and the pressure element 118. The control unit 200 is operable to receive power from an external power source, such as an electrical outlet. the recharge outlet 216 is configured to be in electrical communication with the heat element 114a, 114b, the vibratory element 116, and the pressure element 118.

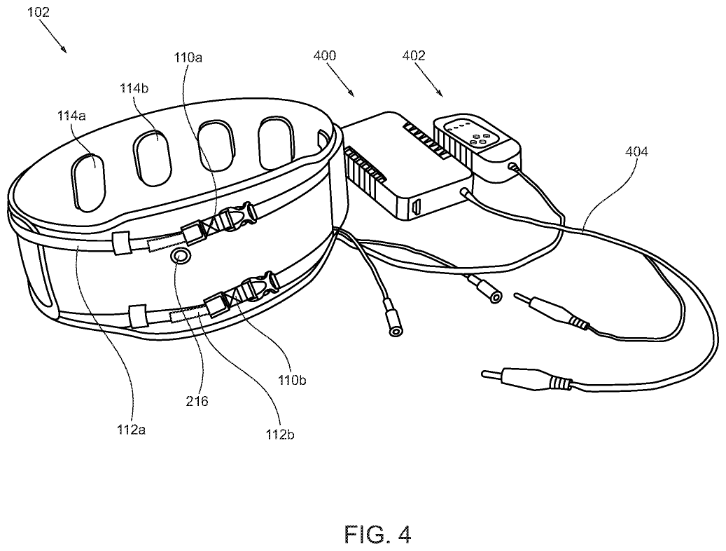

Looking now at FIG. 4, an alternative embodiment of the massage belt comprises a wired control unit 400 connected through multiple wires 404 to the elements 114a-b, 116, 118. Further, the remote controller discussed above is replaced by a wired controller 402, which performs substantially the same function. However, in other embodiments, different combinations of wireless and wired control units and controllers may be used.

In conjunction with the recharge outlet 216, the system 100 provides a power cord 218 that serves to transmit power from the external power source to the recharge outlet. The external power source may include, without limitation, a GFCI outlet, an AFCI outlet, a 20A outlet, a switch outlet, and a USB outlet.

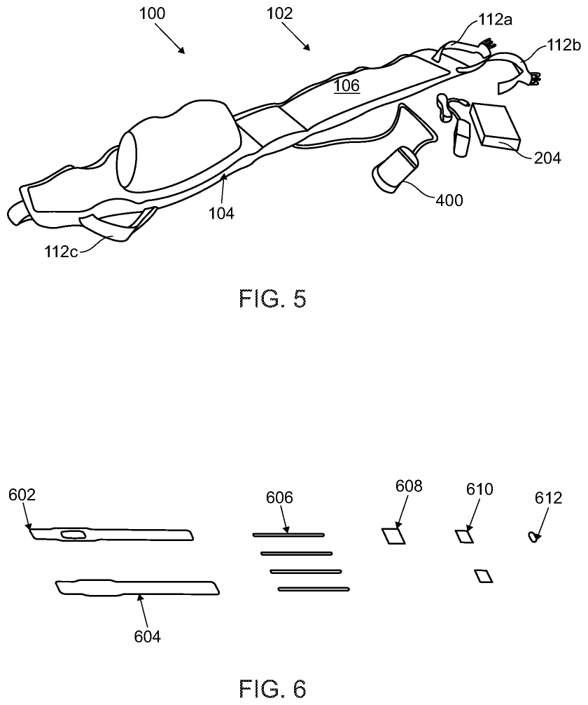

Turning now to FIG. 5, the system 100 provides a remote controller 204 that is in operational communication with the signal reception device 202 of the control unit 200. FIG. 2 illustrates a top view of an exemplary abdomen massaging system, showing the control unit and the remote controller 204. The remote controller 204 is configured to command the control unit 200 to generate the heat signal 220, the vibration signal 222, and the pressure signal 224. This allows the user, or a second person to operate the massage belt 102 directly through the control unit 200, or remotely with the remote controller 204. In some embodiments, the remote controller 204 may include, without limitation, a cell phone, a radio remote controller, and an iPad.

In operation, the user wraps the body portion 104 of the massage belt 102 around the pelvic area 300 (See FIG. 3). A special gel or other massage-related composition may be applied to the pelvic region prior to wearing the massage belt 102. The user may then activate the remote controller 204 to generate at least one of the heat signals 220, vibration signal 222, and pressure signal 224. However, the user may also control the signals from directly on the control unit 200. The intensity and duration of each element can be controlled by the speed level controller 208 and the timer 210. Also, a predetermined time and intensity can be conducted. For example, when the pressure base line is crossed, the pressure element 118 is activated. Or after 30 second durations, activation of each element is alternated. The user can also select between the idle module and the constant module to power on and off the control unit 200.

In an alternative embodiment, an abdomen massaging system 600, shown in FIGS. 6-12 provides a variation of the body portion 104 for massage belt 102 shown above. In this embodiment, body portion comprises an interior wrap 602 and an opposing exterior wrap that are joined in a flush, parallel arrangement (See FIG. 6). Straps 606 still attach to ends of body portion, but are linear and flexible, so as to wrap around body of user, terminating at a hook and loop fastener 610. A pocket 608 is used to contain at least one of the heat element 114a-b, the vibratory element 116, and the pressure element 118. A case 612 is also included. Nonetheless, the abdomen massaging system 600 operates to massage the abdomen 300 through selective application of heat, vibrations, and pressure the muscles of the abdomen for relieving menstrual pains and cramp.

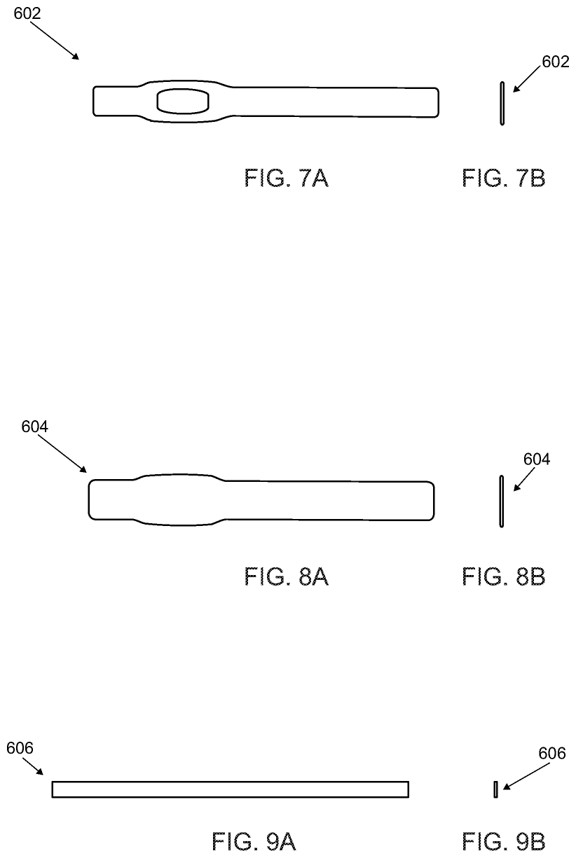

Looking now at FIG. 7A, a top view of the interior wrap 602 and connecting strap 606 is shown. The interior wrap engages the body of the user (See FIG. 7B). FIGS. 8A and 8B illustrates an exterior wrap 604, which orients away from the body, being the visible portion of the massage belt 600. The length of the interior wrap 602 and the exterior wrap 604 may be about 54″. Turning now to FIGS. 9A and 9B, the strap 606 is illustrated. Strap 606 is resilient, and may have fasteners at the end to secure around the body of the user. On exemplary length of strap 606 is about 24″. hook and loop fastener 610, which is shown in FIG. 11A. Further, a side view of hook and loop fastener 610 is illustrated in FIG. 11B.

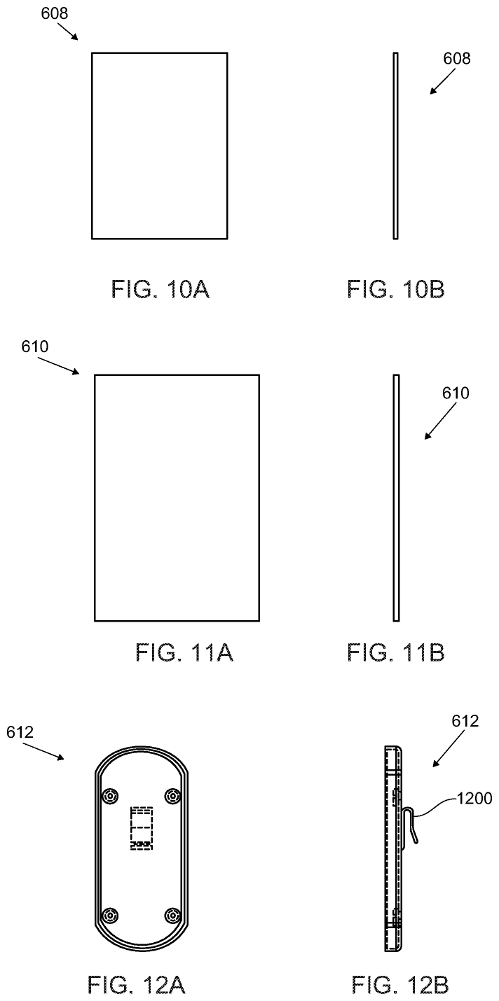

Tuning now to FIGS. 10A-10B, pocket 608 is shown. The pocket 608 may have dimensions of 5.5″×7.5″. However, the pocket 608 may be larger or smaller to accommodate variably sized heat element 114a-b, the vibratory element 116, and the pressure element 118. Turning now to FIG. 12A, a case 612 is illustrated. The case 612 attaches to body portion. Case may also include a clip 1200, as shown inside view of FIG. 12B. However, in other embodiments, other fastening means than a clip may be used.

In conclusion, the abdomen massaging system 100 massages through selective application of heat, vibrations, and pressure the muscles of the abdomen for relieving menstrual pains and cramps. The system utilizes a massage belt that is wearable about the pelvis. The massage belt has a fastener, such as opposing straps, that hold the body portion around the abdomen.

Further, the massage belt 100 is remotely regulated by a control unit or a remote controller to selectively generate vibration with a vibratory element, heat with a heat element, and pressure with a pressure element. These massaging components are controlled by a hands-free control unit that initiates a vibration signal, a heat signal, and a pressure signal to the massage belt. The heat element, vibratory element, and pressure element provide a perceptible stimulus to the pelvic area at predetermined times and intensities. The control unit regulates the signals between a rest mode, a signal mode, a timer, and a speed control.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. An abdomen massaging system, the system comprising:
a massage belt operable to be worn on, or proximal to, the pelvic area, the massage belt comprising a heat element operable to generate heat, the massage belt further comprising a vibratory element operable to generate vibrations, and a pressure element operable to generate pressure;
a control unit operable to regulate the heat element, the vibratory element, and the pressure element,
the control unit comprising a signal transmission device and a signal reception device, the signal transition device transmitting a heat signal to the heat element to initiate heat in the massage belt, the heat signal comprising a heat signal mode and a heat rest mode, the heat signal mode initiating operation of the heat element, the heat rest mode terminating operation of the heat element,
the control unit transmitting a vibration signal to the vibratory element to initiate vibration in the massage belt, the vibration signal comprising a vibration signal mode and a vibration rest mode, the vibration signal mode initiating operation of the vibratory element, the vibration rest mode terminating operation of the vibratory element,
the control unit transmitting a pressure signal to the pressure element to initiate pressure in the massage belt, the pressure signal comprising a pressure signal mode and a pressure rest mode, the pressure signal mode initiating operation of the pressure element, the pressure rest mode terminating operation of the pressure element; and
a remote controller in operational communication with the signal reception device of the control unit, the remote controller operable to command the control unit to generate the heat signal, the vibration signal, and the pressure signal.

2. The system of claim 1, wherein the control unit is disposed on a control side of the belt, and the elements are disposed on a massage side of the belt.

3. The system of claim 1, wherein the heat element comprises a heating pad.

4. The system of claim 1, wherein the vibratory element comprises a vibrator.

5. The system of claim 1, wherein the pressure element comprises a contraction strap or an air pressure bladder.

6. The system of claim 1, wherein the pressure element applies pressure at a base line pressure.

7. The system of claim 6, wherein the pressure signal mode is operative when the pressure applied by the pressure module is above the base line pressure.

8. The system of claim 7, wherein the pressure rest mode is operative below the base line pressure.

9. The system of claim 1, wherein the control unit comprises a speed level controller, the speed level controller being operational to regulate the intensity of the vibratory element and the pressure element.

10. The system of claim 9, wherein the speed level controller comprises a high level, a medium level, and a slow level.

11. The system of claim 1, wherein the control unit comprises a timer, the timer regulating the duration of the signals.

12. The system of claim 1, wherein the control unit comprises a recharge outlet operable to receive power from an external power source, the recharge outlet being in electrical communication with the heat element, the vibratory element, and the pressure element.

13. The system of claim 12, further comprising a power cord carrying power from the external power source to the recharge outlet.

14. The system of claim 1, wherein the control unit comprises an idle module and a constant module, the idle module being operable to terminate operation of the vibratory element, the heat element, and the pressure element.

15. The system of claim 1, wherein the massage belt comprises a body portion and at least one fastener, the fastener being operable to help retain the body portion on, or proximal to, the pelvic area.

16. The system of claim 15, wherein the at least one fastener comprises a pair of straps arranged on the one opposing ends of the body portion to fasten the body portion on the pelvic area.

17. The system of claim 16, wherein the at least one fastener comprises a snap button.

18. The system of claim 1, wherein the remote controller includes at least one of the following: a cell phone, a radio remote controller, and an iPad.

19. An abdomen massaging system, the system comprising:
a massage belt operable to be worn on, or proximal to, the pelvic area, the massage belt comprising a heat element operable to generate heat, the massage belt further comprising a vibratory element operable to generate vibrations, and a pressure element operable to generate pressure, the massage belt further comprising a body portion and at least one fastener, the fastener being operable to help retain the body portion on, or proximal to, the pelvic area;
a control unit joined with the body portion of the massage belt, the control unit operable to regulate the heat element, the vibratory element, and the pressure element,
the control unit comprising a signal transmission device and a signal reception device, the signal transition device transmitting a heat signal to the heat element to initiate heat in the massage belt, the heat signal comprising a heat signal mode and a heat rest mode, the heat signal mode initiating operation of the heat element, the heat rest mode terminating operation of the heat element,
the control unit transmitting a vibration signal to the vibratory element to initiate vibration in the massage belt, the vibration signal comprising a vibration signal mode and a vibration rest mode, the vibration signal mode initiating operation of the vibratory element, the vibration rest mode terminating operation of the vibratory element,
the control unit transmitting a pressure signal to the pressure element to initiate pressure in the massage belt, the pressure signal comprising a pressure signal mode and a pressure rest mode, the pressure signal mode initiating operation of the pressure element, the pressure rest mode terminating operation of the pressure element,
the control unit further comprising a speed level controller, the speed level controller being operational to regulate the intensity of the vibratory element and the pressure element,
the control unit further comprising a timer, the timer regulating the duration of the signals; and
a remote controller in operational communication with the signal reception device of the control unit, the remote controller operable to command the control unit to generate the heat signal, the vibration signal, and the pressure signal.

20. An abdomen massaging system, the system comprising:
a massage belt operable to be worn on, or proximal to, the pelvic area, the massage belt comprising a heat element operable to generate heat, the massage belt further comprising a vibratory element operable to generate vibrations, and a pressure element operable to generate pressure, the massage belt further comprising a body portion and at least one fastener, the fastener being operable to help retain the body portion on, or proximal to, the pelvic area;
a control unit operable to regulate the heat element, the vibratory element, and the pressure element, the control unit being disposed on a control side of the belt, and the elements being disposed on a massage side of the belt,
the control unit comprising a signal transmission device and a signal reception device, the signal transition device transmitting a heat signal to the heat element to initiate heat in the massage belt, the heat signal comprising a heat signal mode and a heat rest mode, the heat signal mode initiating operation of the heat element, the heat rest mode terminating operation of the heat element,
the control unit transmitting a vibration signal to the vibratory element to initiate vibration in the massage belt, the vibration signal comprising a vibration signal mode and a vibration rest mode, the vibration signal mode initiating operation of the vibratory element, the vibration rest mode terminating operation of the vibratory element,
the control unit transmitting a pressure signal to the pressure element to initiate pressure in the massage belt, the pressure signal comprising a pressure signal mode and a pressure rest mode, the pressure signal mode initiating operation of the pressure element, the pressure rest mode terminating operation of the pressure element, the pressure element applying pressure at a base line pressure,
whereby the pressure signal mode is operative when the pressure applied by the pressure module is above the base line pressure,
whereby the pressure rest mode is operative below the base line pressure,
the control unit further comprising a speed level controller, the speed level controller being operational to regulate the intensity of the vibratory element and the pressure element,
the control unit further comprising a timer, the timer regulating the duration of the signals,
the control unit further comprising a recharge outlet operable to receive power from an external power source, the recharge outlet being in electrical communication with the heat element, the vibratory element, and the pressure element; and
a remote controller in operational communication with the signal reception device of the control unit, the remote controller operable to command the control unit to generate the heat signal, the vibration signal, and the pressure signal.
